(19) United States
(12) Patent Application Publication
(10) Pub. No.: US 2006/0275337 A1
(43) Pub. Date: Dec. 7, 2006

(54) Complex coacervate core micelles as surface modification or surface treatment

(76) Inventors: Abraham Martinus Cohen Stuart, Wageningen (NL); Stefan Van Der Burgh, Wagningen (NL); Reint Gerrit Fokkink, Hengelo (NL); Arie De Kreizer, Wageningen (NL)

Correspondence Address: Jean-Louis Seugnet;Rhodia INC., 8 Cedar Brook Drive, Cranbury, NJ, 08512-7500, US

(21) Appl. No.: 10/573,014

(22) PCT Filed: Sep. 24, 2004
(86) PCT No.: PCT/EP04/10773

§ 371 Date: Aug. 9, 2006

(30) Foreign Application Priority Data
Sep. 25, 2003 (EP) 03078031.6

(51) Int. Cl. A61K 31/74; A61F 2/02

(52) U.S. Cl. 424/423; 424/78.09

(57) ABSTRACT

Complex coacervate core micelles as surface modification or surface treatment The present invention relates to devices carrying on the surface polymeric micelles and a process for the preparation thereof. It further relates to the use of polymeric micelles as a surface coating for rendering a surface anti-fouling and/or protein-resistant. The present invention relates to modified or treated surfaces carrying on polymeric micelles and a process for the preparation thereof. It further relates to the use of polymeric micelles as a surface coating for surface modification or surface treatment. The surface modification or surface treatment is for example for rendering a surface anti-fouling and/or protein-resistant, or for preventing bacteria proliferation, disinfecting, suppressing odours, preventing malodour, providing easy-cleaning or soil-release properties. These polymeric micelles are of the so-called complex coacervate core type, exhibiting a hydrophilic, neutral corona and a core, which is formed by charge complexation of oppositely charged blocks.

### FIELD OF INVENTION

The present invention relates to modified or treated surfaces carrying on polymeric micelles and a process for the preparation thereof. It further relates to the use of polymeric micelles as a surface coating for surface modification or surface treatment. The surface modification or surface treatment is for example for rendering a surface anti-fouling and/or protein-resistant, or for preventing bacteria proliferation, disinfecting, suppressing odours, preventing malodour, or for providing easy-cleaning or soil-release properties.

### BACKGROUND

Many devices and materials require properties at the surface to be distinct from the bulk properties of the device or material, particularly in the case of biomedical devices. In order to make these devices biocompatible or prevent non-specific interaction of biopolymers such as plasma proteins and nucleic acids with the surface of the device, their surface properties are improved using coating methods.

Contact lenses are a good example of biomedical devices that require improved surface characteristics. In order to maintain good corneal health they are usually built up from hydrophobic materials that exhibit relatively high oxygen permeability through the bulk of the lens. However, without any surface treatment or modification such a lens will adhere to the eye. Thus a contact lens will generally have a core bulk material that is highly oxygen permeable and hydrophobic, and a surface that has been treated or coated to increase hydrophilic properties. This hydrophilic surface allows the lens to move relatively freely on the eye without adhering excessive amounts of tear lipid and protein.

For the above-mentioned purposes of rendering devices biocompatible, anti-fouling and/or protein non-adsorbing, it is known from the art (see e.g. WO A 99/38858) to coat them with one or more layers of homopolymers, block polymers or mixtures thereof. It is preferred to apply block polymers with the desired hydrophobic and hydrophilic properties combined in one molecule. Coating of these macromolecules is realised either through grafting them onto the surface chemically or by adsorbing them from solution physically.

In WO A 01/32230 it is disclosed how to conveniently use the tendency of those kind of block polymers to self-assemble into regularly shaped micelles upon dissolution in a so-called selective solvent, which is a good solvent for one of the blocks, but a poor one for another. In polar media, such as water, block polymers with hydrophilic and hydrophobic segments aggregate in a way that the hydrophilic corona shields the hydrophobic core from the polar media. In non-polar media, the micelles are reversed. The shape and size of the micelle can be tuned by simply varying either the kind of monomer or the size and proportion of the constituting blocks. Coating of these polymeric micelles with either a hydrophobic core and a hydrophilic shell or the other way around according to WO A 01/32230 yields more defined and uniform layers at the surface than when applying the unassembled macromolecules.

However, the applicability of polymeric micelles with an amphiphilic character is restricted only to a limited number of combinations of surfaces and environment. Not only do the different constituents have to match the properties of the device and be able to yield its biocompatible, anti-fouling or protein non-adsorbing character, but geometry constraints are to be satisfied: self-assembly is induced only if the solvent selectivity among the different parts of the block polymers is sufficient.

Furthermore, like micelles prepared from low molecular weight surfactants, polymeric micelles only form above a so-called critical micelle concentration. Lower concentrations can destruct the micellar coating, which after all consists of physical rather than chemical associations of the amphiphilic molecules.

In WO A 01/32230 these problems are overcome by supplying block polymers with functional end groups through which the micelles chemically attach to the surface and by forming multilayers of micelles with other polymers or reversed micelles. The methods associated therewith are rather laborious, require additional synthesis steps and are still in need of a combination of distinct hydrophobic and hydrophilic blocks.

### BRIEF DESCRIPTION OF THE INVENTION

It is the object of the invention to provide a coating composition comprising polymeric micelles that do not have the above-mentioned drawbacks of the selected group of amphiphilic polymeric micelles. The coating composition according to the invention can be applied to various types of surfaces, using straightforwardly synthesised constituents and is not bothered by a CMC.

It is further an object of the invention to provide with a use of a coating composition comprising at least one polymeric micelle, wherein the polymeric micelle has a hydrophilic, neutral corona and a complex coacervate core, wherein the complex coacervate core is formed by charge complexation, for modifying a surface or treating a surface, for example for rendering at least one surface, for example of a device, protein-resistant, or for preventing bacteria proliferation, disinfecting, suppressing odours, preventing malodour, or for providing easy-cleaning or soil-release properties.

### DEFINITIONS

In the present application, a “coating”, is understood, by any treatment or modification leaving at least one chemical compound onto a surface. Thus a coating can be understood as a layer of a composition, or as a deposition of at least one chemical compound.

The term “complex coacervation”, as used herein, refers to the interaction of two macromolecules of opposite charge. In the literature coacervation addresses the separation of colloidal systems in liquid phases, wherein the phase more concentrated in hydrophilic colloid component is called the coacervate. The term “complex” indicates that the driving force for separation of colloids is of electrostatic origin. Micelles that are formed by the principle of complex coacervation are called complex coacervate core micelles (CCCM).

In the context of the polymeric micelles of the invention complex coacervation is sometimes otherwise referred to as complex flocculation, block ionomer complexation (BIC), polyelectrolyte complexation (PEC) or even interpolyelectrolyte complexation (IPEC).

In the present specification, a unit deriving from a monomer is understood as a unit that may be directly obtained from the said monomer by polymerizing. Thus, a unit deriving from an ester of acrylic or methacrylic acid does not encompass a unit of formula —H—CH(COOH)—, —H—C(CH₃)(COOH)—, —CH—CH(OH)—, —CH—C(CH₃)(OH)—, obtained for example by polymerizing an ester of acrylic or methacrylic acid, or a vinyl acetate, and then hydrolyzing. A unit deriving from acrylic acid or methacrylic acid encompasses for example a unit obtained by polymerizing a monomer (for example an alkyl acrylate or methacylate) and then reacting (for example hydrolyzing) to obtain units of formula —CH—CH(COOH)— or —CH—C(CH₃)(COOH)—. A unit deriving from vinyl alcohol encompasses for example a unit obtained by polymerizing a monomer (for example a vinyl ester) and then reacting (for example hydrolyzing) to obtain units of formula —CH—CH(OH)— or —CH—C(CH₃)(OH)—.

In the present specification, the molecular weight of a polymer, copolymer, or block refers to the weight-average molecular weight of said polymer, copolymer, part or block. The weight-average molecular weight of the polymer or copolymer can be measured by gel permeation chromatography (GPC). In the present specification, the molecular weight of a block refers to the molecular weight calculated from the amounts of monomers, polymers, initiators and/or transfer agents used to make the said block. The one skilled in the art knows how to calculate these molecular weights. The ratios by weight between blocks refer to the ratios between the amounts of the compounds used to make said parts or blocks, considering an extensive polymerization.

Typically, the molecular weight M of a block is calculated according to the following formula:

$$M = {\sum }_{i} {M}_{i} * \frac{{n}_{i}}{{n}_{precursor}} ,$$

wherein Mᵢ is the molecular weight of a monomer i, nᵢ is the number of moles of a monomer i, and n_{precursor} is the number of moles of a compound the macromolecular chain of the block will be linked to. Said compound may be a transfer agent or a transfer group, or a previous block. If it is a previous block, the number of moles maybe considered as the number of moles of a compound the macromolecular chain of said previous block has been linked to, for example a transfer agent or a transfer group. It may be also obtained by a calculation from a measured value of the molecular weight of said previous block. If two blocks are simultaneously grown from a previous block, for example at ends, the molecular weight calculated according to the above formula should be divided by two.

### DETAILED DESCRIPTION OF THE INVENTION

The essential parts of the complex coacervate core micelles are a charged block, a second charged block with opposite charge, and a hydrophilic and neutral block. All constituents exhibit excellent water solubility.

In one embodiment of the invention these essential features are comprised in a single polymer, preferably a triblock terpolymer with an anionic block, a cationic block and a neutral hydrophilic block, in any order. However, in a preferred embodiment the polymeric micelle according to the invention comprises at least a first and a second polymer.

The first and the second polymer are oppositely charged, meaning that one polymer exhibits an overall anionic character and the other polymer has an overall cationic character. The ionic properties can be accounted for by groups that are permanently charged or “quenched” in aqueous environment, such as e.g. sulfonated groups, or by chargeable or “annealed” groups which are dependent on pH, such as e.g. amines and carboxylated groups. From hereon, the term “chargeable”, as used herein, refers to both permanently charged groups and chargeable groups. It can be also referred to “ionic” (charged) units, monomers, blocks, or polymers, or to “potentially ionic” (chargeable) units, monomers, blocks, or polymers. For the sake of simplification, in the present specification, “ionic” (responsibly anionic or cationic) refers to both ionic and potentially ionic (responsibly potentially anionic or potentially cationic), unless otherwise specified.

The first polymer is a block polymer with an ionic block comprising at least 6, more preferably at least 20 and most preferably at least 40 chargeable groups. The term “block polymer”, as used herein, refers to polymers that are constructed from blocks of more than one monomer (or units deriving from monomer(s)). It does not comprise polymers with random distributions of more than one monomer. However a block can be itself a random distribution of units deriving from more than one monomer. According to the invention it is preferred that the block polymer is a diblock copolymer with end-to-end chains or a triblock terpolymer with a third block in-between. Advantageously, at least one block, preferably at least two, comprise units deriving from mono-alpha-ethylenically-unsatured monomers.

The term “ionic block”, as used herein, is meant to include blocks of charged groups and chargeable groups. The block polymer can be overall cationically or anionically chargeable. Preferably an ionic block with a sequence of at least 6, more preferably at least 20, and with most preference at least 40 chargeable groups can be employed for the polymeric micelles according to the invention.

The ionic block of the block polymer is preferably selected from the group consisting of poly-L-lysine or other poly(amino acids), polyacrylic acid (PAA), polymethacrylic acid (PMA), DNA-segments, poly(thiophene-3-acetic acid), poly(4-styrenesulfonic acid), polyvinylpyrrolidone, poly(pyridinium acetylene), poly(ethylene imine), poly(vinylbenzyltriamethylamine), polyaniline, polypyrrole, poly(alkylamine hydrochloride), poly-(dimethylamino ethylmethacrylate) (PAMA), polyaspartic acid, poly(N-alkyl-4-vinylpyridinium) (PVP), but it is not limited thereto. More preferably, the ionic block is selected from the group of polyacrylic-acid (PAA), polymethacrylic acid (PMA), poly-(dimethylamino ethylmethacrylate) (PAMA) and poly(N-alkyl-4-vinylpyridinium) PVP).

The block can also be defined by the units it comprises and/or by the monomers the units derive from. Thus the ionic block can comprise anionic units or cationic units.

Examples of cationic blocks are blocks comprising units deriving from cationic monomers such as:

aminoalkyl(meth)acrylates, aminoalkyl(meth)acrylamides,

monomers, including particularly (meth)acrylates, and (meth)acrylamides derivatives, comprising at least one secondary, tertiary or quaternary amine function, or a heterocyclic group containing a nitrogen atom, vinylamine or ethylenimine;

diallyldialkyl ammonium salts;

their mixtures, their salts, and macromonomers deriving from therefrom.

Examples of cationic monomers include:

dimethylaminoethyl(meth)acrylate, dimethylaminopropyl(meth)acrylate, ditertiobutylaminoethyl(meth)acrylate, dimethylaminomethyl(meth)acrylamide, dimethylaminopropyl(meth)acrylamide;

ethylenimine, vinylamine, 2-vinylpyridine, 4-vinylpyridine;

trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido (also called 2-(acryloxy)ethyltrimethylammonium, TMAEAMS) chloride, trimethylammonium ethyl(meth)acrylate (also called 2-(acryloxy)ethyltrimethylammonium, TMAEAMS) methyl sulphate, trimethyl ammonium propyl(meth)acrylamido chloride, vinylbenzyl trimethyl ammonium chloride,

diallyldimethyl ammonium chloride,

monomers having the following formula: 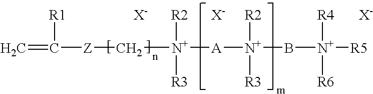
wherein
R₁ is a hydrogen atom or a methyl or ethyl group;
R₂, R₃, R₄, R₅ and R₆, which are identical or different, are linear or branched C₁-C₆, preferably C₁-C₄, alkyl, hydroxyalkyl or aminoalkyl groups;
m is an integer from 1 to 10, for example 1;
n is an integer from 1 to 6, preferably 2 to 4;
Z represents a —C(O)O— or —C(O)NH— group or an oxygen atom;
A represents a (CH₂)ₚ group, p being an integer from 1 to 6, preferably from 2 to4;
B represents a linear or branched C₂-C₁₂, advantageously C₃-C₆, polymethylene chain optionally interrupted by one or more heteroatoms or heterogroups, in particular O or NH, and optionally substituted by one or more hydroxyl or amino groups, preferably hydroxyl groups;
X, which are identical or different, represent counterions, and
   their mixtures, and macromonomers deriving therefrom.

Examples of anionic blocks are blocks comprising units deriving from anionic monomers selected from the group consisting of:

alpha-ethylenically-unsaturated, preferably mono-alpha-ethylenically-unsaturated, monomers comprising a phosphate or phosphonate group,

alpha-ethylenically-unsaturated, preferably mono-alpha-ethylenically-unsaturated, monocarboxylic acids,

monoalkylesters of alpha-ethylenically-unsaturated, preferably mono-alpha-ethylenically-unsaturated, dicarboxylic acids,

monoalkylamides of alpha-ethylenically-unsaturated, preferably mono-alpha-ethylenically-unsaturated, dicarboxylic acids,

alpha-ethylenically-unsaturated, preferably mono-alpha-ethylenically-unsaturated, compounds comprising a sulphonic acid group, and salts of alpha-ethylenically-unsaturated compounds comprising a sulphonic acid group.

Preferred anionic blocks include blocks comprising units deriving from at least one anionic monomer selected from the group consisting of:

acrylic acid, methacrylic acid,

vinyl sulphonic acid, salts of vinyl sulfonic acid,

vinylbenzene sulphonic acid, salts of vinylbenzene sulphonic acid,

alpha-acrylamidomethylpropanesulphonic acid, salts of alpha-acrylamidomethylpropanesulphonic acid

2-sulphoethyl methacrylate, salts of 2-sulphoethyl methacrylate,

acrylamido-2-methylpropanesulphonic acid (AMPS), salts of acrylamido-2-methylpropanesulphonic acid, and

styrenesulfonate (SS).

The first polymer further comprises at least a hydrophilic and neutral block. If the first polymer is a linear polymer, the blocks are end-to-end linked. If the first polymer is branched, the chargeable groups could be grafted onto a hydrophilic and neutral backbone or vice versa. However, it is preferred that the first polymer is a linear chain.

In principle any hydrophilic and neutral block can be employed, the only restrictions being that it is water-soluble, can be connected to the ionic block and exhibits the desired features for the surface of the device. The hydrophilic and neutral block of the block polymer can be selected from the group consisting of polyethylene glycol (PEG), polyglyceryl methacrylate (PGMA), polyvinylalcohol, polyacrylamide (PAM), polymethacrylamide, but it is not limited thereto. It is preferred that the hydrophilic and neutral block has protein-resistant and/or anti-fouling properties. More preferably, the hydrophilic and neutral block is a polyethylene glycol (PEG) or a polyacrylamide (PAM), or a combination thereof.

The block can also be defined by the units it comprises and/or by the monomers the units derive from. Thus the ionic block can comprise neutral hydrophilic units.

Examples of neutral hydrophilic blocks are blocks comprising units deriving from neutral hydrophilic monomers, selected from the group consisting of:

ethylene oxide,

vinyl alcohol,

vinyl pyrrolidone,

acrylamide, methacrylamide,

polyethylene oxide(meth)acrylate (i.e. polyethoxylated(meth)acrylic acid),

hydroxyalkylesters of alpha-ethylenically-unsaturated, preferably mono-alpha-ethylenically-unsaturated, monocarboxylic acids, such as 2-hydroxyethylacrylate, and

hydroxyalkylamides of alpha-ethylenically-unsaturated, preferably mono-alpha-ethylenically-unsaturated, monocarboxylic acids.

The hydrophilic and neutral block of the first polymer preferably has a molecular weight of less than 10,000 K, more preferably less than 5,000 K, most preferably less than 1,000 K.

The second polymer according to the invention can be a homopolymer, a random copolymer, a block polymer, a natural polymer, or a derivative thereof. In case the second polymer is a homopolymer, or a random copolymer it is a polyelectrolyte. If the second polymer is a block polymer, it comprises at least an ionic block. The polyelectrolyte or the ionic block can be either cationically or anionically chargeable, wherein it is charged oppositely to the ionic block of the first polymer. If the ionic block of the first polymer is a polycation, than the second polymer has an overall anionic character, and if the ionic block of the first polymer is a polyanion, than the second polymer has an overall cationic character. If the second polymer is a random copolymer, it preferably comprises a combination of anionic units, or a combination of cationic units, or a combination of anionic units and neutral units, or a combination of cationic units and neutral units.

The second polymer according to the invention can be a homopolymer or a random copolymer, a block polymer, a natural polymer, or a derivative thereof. It can be chosen from the same group of polyanions and polycations as the ionic block of the first polymer, but is not limited to this list. In other words, the second polymer, or block thereof, can comprise units and/or derive from monomers, that have been listed above, provided that it is cationic where the first polymer has an anionic block, or that it is anionic where the first polymer has a cationic block. In principle, any ionic block can be used in the preparation of the complex coacervate core micelles according to the invention, the only restraint being that it is oppositely chargeable to the ionic block of the first polymer. Most preferably, the second polymer is selected from the group of polyacrylic acid (PAA), polymethacrylic acid (PMA), poly(dimethylamino ethylmethacrylate) (PAMA) and poly(N-alkyl-4-vinylpyridinium) (PVP). It can be also a homopolymer or a random copolymer comprising units deriving from the monomers listed above.

In case the second polymer is a homopolymer, it preferably consists of at least 50, more preferably at least 200, and most preferably at least 500 monomeric units. The homopolymer has a molecular weight of preferably less than 100,000 K, more preferably less than 50,000 K, most preferably less than 10,000 K.

In case the second polymer is a block polymer, the ionic block preferably consists of at least 50, more preferably at least 200, and most preferably at least 500 monomeric units. The ionic block of the second polymer has a molecular weight of preferably less than 100,000 K, more preferably less than 50,000 K, most preferably less than 10,000 K.

In case the second polymer is a block polymer, the second block can be a neutral block that is the same or different than the second block of the first polymer. In principle any monomer can be applied, the only restrictions being that a block of these monomers is water-soluble, can be connected to the ionic block and exhibits the desired features for the surface of the device. It is preferred that the hydrophilic and neutral block has protein-resistant and/or anti-fouling properties. Most preferably, the hydrophilic and neutral block is a polyethylene glycol, a polyglyceryl methacrylate or a polyacrylamide, or a combination thereof.

It is an object of the invention to provide with a use of a coating composition for the reduction or prevention of protein adsorption and/or anti-fouling. The coating composition according to the invention further relates to surface modification or surface treatment.

The surface modification or surface treatment can be the reduction or prevention of protein adsorption and/or the anti-fouling.

The surface modification or surface treatment can be preventing bacteria proliferation, disinfecting, suppressing odours, preventing malodour, or providing easy-cleaning or soil-release properties.

The composition can be for example is a home-care or fabric-care or institutional-cleaning or industrial-cleaning composition. The composition can be for example a paint or a sealant, such as a boat paint. Further details about some compositions and/or surface treatments or surface modifications and/or specific surfaces, optionally of devices, will be given below.

The present invention also relates to the use of the complex coacervate core micelles for protein purification and drug and gene delivery. It is preferred to use the coating composition according to the invention to coat at least one surface of a device.

It is further an object of the invention to provide with a process for modifying or treating a surface, for example for coating the surface of a device, said process comprising:
(i) mixing at least a first and a second polymer in such amounts that the resulting mixture has a fraction of the total number of cationic polymeric groups over the total number of charged groups in the range of 0.2 to 0.8, wherein the first and the second polymer are oppositely charged and wherein the first polymer is a block polymer comprising at least a hydrophilic and neutral block; and
(ii) bringing the resulting mixture in contact with the surface of a device,
   wherein the salt concentration in both steps is less than 1 M, more preferably less than 0.2 M and most preferably less than 0.05 M.

Upon mixing of the first and the second polymer in step (i), the ionic groups of the first polymer and the oppositely charged ionic groups on the second polymer induce complex coacervation, leading to a soluble complex without excess charge, as the charges are all restricted to the complex domain that is formed by the coacervate core. The hydrophilic and neutral block(s) form(s) the corona The size of the core is determined by the amount of charged groups, whereas the size of the corona is entirely dependent on the amount of neutral monomers. The polymeric micelles according to the invention are self-assemblies that are not induced by amphiphilicity or solvent selectivity, and their constituents all exhibit excellent water solubility.

It is thought that the driving forces for this type of phase separation are the entropy gain connected with the liberation of small counterions that were initially confined by the electric field of the participating ionic blocks and the decrease in the electrostatic energy of the ionic blocks due to a more efficient screening of the charges.

The self-assemblies according to the invention lack a CMC. The formation of complex coacervate core micelles is therefore not restricted to the length ratio of the chargeable and neutral blocks and does not have a minimal concentration in which the constituents need to be supplied with.

However, it is found that the formation of the complex coacervate core micelles is rather dependent on the total number of anionically and cationically chargeable groups. In order to define the conditions to work the invention, the composition is not expressed in terms of the fraction of monomeric units, but as the fraction of cationic groups f⁺

$${f}^{+} = \frac{number of cationic groups}{total number of charged groups}$$

or the fraction of anionic groups f=1-f⁺.

There is a limiting range in f⁺ in which self-assembly into polymeric micelles takes place, around the so-called preferred micellar composition (PMC), which relates to the relative amounts of cationic and anionic groups in the composition. Outside this range no complex coacervate core micelles are formed. The PMC does not form a lower limit like the CMC known from amphiphilic micelles, but rather an optimum in a range with a lower and an upper limit. The PMC is found to be at f⁺=0.5, when the total amount of positively charged groups more or less equals the total amount of negatively charged groups in the composition. It is preferred that f⁺ is in the range from 0.2 to 0.8, with more preference from 0.3 to 0.7 and with most preference from 0.4 to 0.6.

In an embodiment of the invention the polymeric micelle comprises more than one of the first polymer and more than one of the second polymer, wherein the relative amounts are governed by the above-mentioned restriction on f⁺. The so-called aggregation number relates to the number of polymers that are involved in micellisation.

The aggregation number of the micelles decreases strongly with increasing length of the corona block resulting in a small increase in micellar radius. Therefore the ratio of the core and corona block sizes are reflected in the thickness of the complex coacervate layer attached to the surface and the layer of neutral polymer brushes. Core and corona block lengths are parameters that a person skilled in the art can vary to tune the coating properties to the required application, i.e. binding strength and e.g. antifouling properties respectively.

The micellisation occurrence is determined by (i) the block length ratio; (ii) the total block length of the block polymer; (iii) the chemical structure (i.e. the hydrophilicity) of the corona monomers; and (iv) the molecular weight and type of ionic groups of the second polymer. Stable micelles are only formed if the length of the ionic block and the hydrophilic and neutral block on the block polymer are appropriate.

If the neutral block is too short compared with the charged parts, the system substantially degrades to a mixture of oppositely charged polyelectrolytes and leads to macroscopic phase separation rather than the formation of micelles. However, it is found that too high a ratio of the amount of hydrophilic and neutral monomers over the amount of charged groups does also not lead to the micelles according to the invention. It is therefore preferred to keep the ratio of the number of hydrophilic and neutral monomers over the number of charged groups on the first block polymer in the range of 1 to 10, with more preference in a range of 2 to 9, and with most preference in the range of 3 and 8. If the ratio exceeds 3 precipitation can be avoided completely. This ratio also relates to the hydrophilicity of the hydrophilic and neutral block: When the hydrophilic properties improve, a smaller ratio suffices for micellisation.

In one embodiment of the invention it is preferred to have the ratio in the range of 3 to 10 if the number of charged groups involved in complexation is less than 14, preferably less than 35.

As mentioned above, it is found that in the case of very asymmetric block length ratios, where the corona is much longer than the core block, no micelles are formed. For the invention to work it is therefore preferred that the second polymer has at least 20, more preferably at least 100, and most preferably at least 500 charged monomeric units.

It is further found that complex coacervation is entirely suppressed if the salt concentration reaches a critical value. If the ionic strength in the solutions containing the polyelectrolytes is too high, no complexation will take place upon mixing. Upon addition of salt to a complex coacervation system, the coacervate phase will redissolve into its separate constituents. It is therefore preferred that during the mixing of step (i) and the coating of step (ii) the salt concentration is less than 1 M, more preferably less than 0.2 M and most preferably less than 0.05 M.

It is further an object of the invention to provide with a modified surface or treated surface, for example a coated device (the surface being the surface of the device), obtainable by a process as described above.

It is also an object of the invention to provide with a modified surface or treated surface, for example a coated device (the surface being the surface of the device), in which at least one polymeric micelle is physically bonded to the, for example to at least one surface of the device. The physical bonding between the micelles and the surface is preferably achieved by adsorption from solution. Preferably the surface is dipped into the solution obtained from step (i) for a time sufficient for the polymeric micelles to adsorb from the solution onto the surface, thereby preferably forming a monolayer. The coating application according to the invention may be accomplished in a number of ways which are known by a person having ordinary skill in the art.

The properties of the surface to be treated or modified can be of any nature, either hydrophobic or hydrophilic, negatively or positively charged. Without any limitation thereto, a device or apparatus made of metal, metal alloys, ceramics, glasses, silica, wood and polymeric materials and the like may be coated according to this invention.

It is preferred to use the coating composition according to the invention for the coating of a biomedical device, including a wide variety of devices used in the biological, medical or personal care industries, especially those requiring contact with blood or other protein-containing fluids, or requiring contact with tissues. Such applications may be found in externally used artificial organs or extracorpeal therapeutic devices such as, for example, kidney dialysis and hemoperfusion devices as well as implantable or partially implantable artificial organs or devices such as vascular access devices, insulin pump tubing, urinary or venous catheters, etc. In addition, other portions of artificial organ devices may be coated. In an implantable device, for example, the entire external surface area may be coated to increase the device's biocompatibility. All internal blood-contacting portions of a device may be coated to reduce protein binding, thereby reducing or eliminating thrombogenicity.

Other medical devices may be coated, as may various types of labware which is used in conjunction with tissue or cell cultures, protein-containing fluids such as blood or serum, or the like. This would include, but not be limited to, assay plates, supports or membranes, glassware, cell culture or bioreactor devices or assemblies, tubing for blood transfer, blood cell storage bags, filters, pharmaceutical manufacturing and packaging, protein isolation, preparation and purification devices or systems, etc. In addition, woven or non-woven cloth or cloth-like materials used in laboratory or medical settings may be coated or impregnated with the polymers of this invention to increase resistance to protein binding, thereby reducing staining from protein sources. Coated articles prepared according to this invention will be particularly useful for re-usable systems, devices, etc., in order to minimise cross-contamination and to facilitate protein removal by washing.

The home-care, or fabric-care composition can be a hard surface cleaner or detergent composition.

Up and below, “composition” refers to a composition that can be used as such, or to a composition that is to be diluted before or during use (it can be referred to a “concentrate composition”) or to a composition in a diluted form (it can be referred to a “diluted composition”).

Up and below, “hard surface” refers to any surface found in homes or institutional buildings, or in industrial buildings, or in furniture, provided that the surface is not a fabric such as clothes, furniture fabrics, curtains, sheets. Hard surfaces especially include dishware, silverware, and any other surfaces that need to be cleaned regularly.

The composition can be in different forms. The form usually depends upon the use of the composition. Thus the composition can be a liquid, a gel, a foaming liquid, a foam, a powder, or a tablet.

Liquid compositions can be dispensed for example by spraying, by pouring or by applying with a liquid dispensing device such as rollers or pumps.

The composition can be diluted prior to being used, or during use. Dilution is preferably performed with water. Dilution rates usually depend upon the use of the composition. Furthermore, dilutions approaches can depend upon the very consumer that uses the composition.

Where the composition is a liquid composition, for example a liquid concentrate composition, said composition can comprise a liquid medium, preferably an aqueous medium, an alcoholic medium, or a hydroxy-alcoholic medium. The medium can be a part of the composition, part of a concentrate composition, or part of a diluted composition, for example the diluting medium.

The composition usually comprises further ingredients such as ingredients of home-care or fabric-care or institutional-cleaning or industrial-cleaning compositions. The further ingredients usually depend upon the use, destination and/or form of the composition. These further ingredients are known by the one skilled in the art of preparing compositions comprising several ingredients (or formulation) to serve a use or a market.

Thus, the composition can comprise at least one surfactant. Compositions that comprise a surfactant are for example useful in compositions that are to provide cleaning or degreasing, such as dish-washing compositions (hand dishwashing or automatic dishwashing), laundry compositions, hard-surface cleaning compositions, industrial cleaning or degreasing compositions.

Useful surfactants include anionic, non ionic, cationic, amphoteric (including zwitterionic) surfactants and mixture thereof.

Anionic Surfactants

Anionic surfactants useful in the present invention are preferably selected from the group consisting of, linear alkylbenzene sulfonate, alpha olefin sulfonate, paraffin sulfonates, methyl ester sulfonates, alkyl sulfates, alkyl alkoxy sulfate, alkyl sulfonates, alkyl alkoxy carboxylate, alkyl alkoxylated sulfates, sarcosinates, taurinates, and mixtures thereof.

One type of anionic surfactant which can be utilized encompasses alkyl ester sulfonates. These are desirable because they can be made with renewable, nonpetroleum resources. Preparation of the alkyl ester sulfonate surfactant component can be effected according to known methods disclosed in the technical literature. For instance, linear esters of C₈-C₂₀ carboxylic acids can be sulfonated with gaseous SO₃ according to “The Journal of the American Oil Chemists Society,” 52 (1975), pp. 323-329. Suitable starting materials would include natural fatty substances as derived from tallow, palm, and coconut oils, etc.

The preferred alkyl ester sulfonate surfactant, especially for laundry applications, comprises alkyl ester sulfonate surfactants of the structural formula: 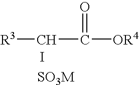
wherein R³ is a C₈-C₂₀ hydrocarbyl, preferably an alkyl, or combination thereof, R⁴ is a C₁-C₆ hydrocarbyl, preferably an alkyl, or combination thereof, and M is a soluble salt-forming cation. Suitable salts include metal salts such as sodium, potassium, and lithium salts, and substituted or unsubstituted ammonium salts, such as methyl-, dimethyl, -trimethyl, and quaternary ammonium cations, e.g. tetramethyl-ammonium and dimethyl piperdinium, and cations derived from alkanolamines, e.g. monoethanol-amine, diethanolamine, and triethanolamine.

Preferably, R³ is C₁₀-C₁₆ alkyl, and R⁴ is methyl, ethyl or isopropyl. Especially preferred are the methyl ester sulfonates wherein R³ is C₁₄-C₁₆ alkyl.

Alkyl sulfate surfactants are another type of anionic surfactant of importance for use herein. In addition to providing excellent overall cleaning ability when used in combination with polyhydroxy fatty acid amides (see below), including good grease/oil cleaning over a wide range of temperatures, wash concentrations, and wash times, dissolution of alkyl sulfates can be obtained, as well as improved formulability in liquid detergent formulations are water soluble salts or acids of the formula ROSO₃M wherein R preferably is a C₁₀-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a cation, e.g., an alkali or alkaline (Group IA or Group IIA) metal cation (e.g., sodium, potassium, lithium, magnesium, calcium), substituted or unsubstituted ammonium cations such as methyl-, dimethyl and trimethyl ammonium and quaternary ammonium cations, e.g., tetramethylammonium and dimethyl piperdinium, and cations derived from alkanolamines such as ethanolamine, diethanolamine, triethanolamine, and mixtures thereof, and the like. Typically, alkyl chains of C₁₂-C₁₆ are preferred for lower wash temperatures (e.g., below about 50° C.) and C₁₆-C₁₈ alkyl chains are preferred for higher wash temperatures (e.g., above about 50° C.). Examples of these surfactants include surfactants sold by Rhodia under the Rhodapan Trade Name.

Alkyl alkoxylated sulfate surfactants are another category of useful anionic surfactant. These surfactants are water soluble salts or acids typically of the formula RO(A)ₘSO₃M wherein R is an unsubstituted C₁₀-C₂₄ alkyl or hydroxyalkyl group having a C₁₀-C₂₄ alkyl component, preferably a C₁₂-C₂₀ alkyl or hydroxyalkyl, more preferably C₁₂-C₁₈ alkyl or hydroxyalkyl, A is an ethoxy or propoxy unit, m is greater than zero, typically between about 0.5 and about 6, more preferably between about 0.5 and about 3, and M is H or a cation which can be, for example, a metal cation (e.g., sodium, potassium, lithium, calcium, magnesium, etc.), ammonium or substituted-ammonium cation. Alkyl ethoxylated sulfates as well as alkyl propoxylated sulfates are contemplated herein. Specific examples of substituted ammonium cations include methyl-, dimethyl-, trimethyl-ammonium and quaternary ammonium cations, such as tetramethyl-ammonium, dimethyl piperidinium and cations derived from alkanolamines, e.g. monoethanolamine, diethanolamine, and triethanolamine, and mixtures thereof. Exemplary surfactants are C₁₂-C₁₈ alkyl polyethoxylate (1.0) sulfate, C₁₂-C₁₈ alkyl polyethoxylate (2.25) sulfate, C₁₂-C₁₈ alkyl polyethoxylate (3.0) sulfate, and C₁₂-C₁₈ alkyl polyethoxylate (4.0) sulfate wherein M is conveniently selected from sodium and potassium. Surfactants for use herein can be made from natural or synthetic alcohol feedstocks. Chain lengths represent average hydrocarbon distributions, including branching. Examples of these surfactants include surfactants sold by Rhodia under the Rhodapex Trade Name.

Other Anionic Surfactants—Other anionic surfactants useful for detersive purposes can also be included in the compositions hereof. These can include salts (including, for example, sodium, potassium, ammonium, and substituted ammonium salts such as mono-, di- and triethanolamine salts) of soap, C₈-C₂₀ linear alkylbenzenesulphonates, for example sold by Rhodia under the Rhodacal trande name, C₈-C₂₂ primary or secondary alkanesulphonates, C₈-C₂₄ olefinsulphonates, sulphonated polycarboxylic acids prepared by sulphonation of the pyrolyzed product of alkaline earth metal citrates, e.g., as described in British patent specification No. 1,082,179, alkyl glycerol sulfonates, fatty acyl glycerol sulfonates, fatty oleyl glycerol sulfates, alkyl phenol ethylene oxide ether sulfates, paraffin sulfonates, alkyl phosphates, isothionates such as the acyl isothionates, N-acyl taurates, fatty acid amides of methyl tauride, alkyl succinamates and sulfosuccinates, monoesters of sulfosuccinate, for example sold by Rhodia under the Geropon trade name (especially saturated and unsaturated C₁₂-C₁₈ monoesters) diesters of sulfosuccinate (especially saturated and unsaturated C₆-C₁₄ diesters), N-acyl sarcosinates, sulfates of alkylpolysaccharides such as the sulfates of alkylpolyglucoside (the nonionic nonsulfated compounds being described below), branched primary alkyl sulfates, alkyl polyethoxy carboxylates such as those of the formula RO(CH₂CH₂O)ₖCH₂COO⁻M+ wherein R is a C₈-C₂₂ alkyl, k is an integer from 0 to 10, and M is a soluble salt-forming cation, and fatty acids esterified with isethionic acid and neutralized with sodium hydroxide. Resin acids and hydrogenated resin acids are also suitable, such as rosin, hydrogenated rosin, and resin acids and hydrogenated resin acids present in or derived from tall oil.

Further examples are given in “Surface Active Agents and Detergents” (Vol. I and II by Schwartz, Perry and Berch). A variety of such surfactants are also generally disclosed in U.S. Pat. No. 3,929,678, issued Dec. 30, 1975 to Laughlin, et al. at Column 23, line 58 through Column 29, line 23.

Nonionic Surfactants

Suitable nonionic detergent surfactants are generally disclosed in U.S. Pat. No. 3,929,678, Laughlin et al., issued Dec. 30, 1975, at column 13, line 14 through column 16, line 6, incorporated herein by reference. Exemplary, non-limiting classes of useful nonionic surfactants include: alkyl dialkyl amine oxide, for example sold by Rhodia under the Rhodamox trade name, alkyl ethoxylate, for example sold by Rhodia under the Rhodasurf trade name, alkanoyl glucose amide, alkyl betaines, for example sold by Rhodia under the Mirataine trade name, and mixtures thereof. Other nonionic surfactants for use herein include:

The polyethylene, polypropylene, and polybutylene oxide condensates of alkyl phenols. In general, the polyethylene oxide condensates are preferred. These compounds include the condensation products of alkyl phenols having an alkyl group containing from about 6 to about 12 carbon atoms in either a straight chain or branched chain configuration with the alkylene oxide. In a preferred embodiment, the ethylene oxide is present in a amount equal to from about 5 to about 25 moles of ethylene oxide per mole of alkyle phenol. Commercially available nonionic surfactants of this type include surfactants sold by Rhodia under the Igepal trade name. These are commonly referred to as phenol alkoxylates, (e.g., alkyl phenol ethoxylates).

The condensation products of aliphatic alcohols with from about 1 to about 25 moles of ethylene oxide. The alkyl chain of the aliphatic alcohol can either be straight or branched, primary or secondary, and generally contains from about 8 to about 22 carbon atoms. Particularly preferred are the condensation products of alcohols having an alkyl group containing from about 10 to about 20 carbon atoms with from about 2 to about 18 moles of ethylene oxide per mole-of alcohol.

Examples of commercially available nonionic surfactants of this type include TergitolB 15-S-9 (the condensation product of C₁₁-C₁₅ linear secondary alcohol with 9 moles ethylene oxide), Tergitol 24-L-6 NMW (the condensation product of C₁₂-C₁₄ primary alcohol with 6 moles ethylene oxide with a narrow molecular weight distribution), both marketed by Union Carbide Corporation; Neodol® 45-9 (the condensation product of C₁₄-C₁₅ linear alcohol with 9 moles of ethylene oxide), Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 45-4 (the condensation product of C₁₄-C₁₅ linear alcohol with 4 moles of ethylene oxide), marketed by Shell Chemical Company, Rhodasurf IT, DB, and B marketed by Rhodia, Plurafac LF 403, marketed by BASF, and Kyro® EOB (the condensation product of C₁₃-C₁₅ alcohol with 9 moles ethylene oxide), marketed by The Procter & Gamble Company. Other commercially available nonionic surfactants include Dobanol 91-8® marketed by Shell Chemical Co. and Genapol UD-080® marketed by Hoechst. This category of nonionic surfactant is referred to generally as “alkyl ethoxylates.”

The condensation products of ethylene oxide with a hydrophobic base formed by the condensation of propylene oxide with propylene glycol. The hydrophobic portion of these compounds preferably has a molecular weight of from about 1500 to about 1800 and exhibits water insolubility. The addition of polyoxyethylene moieties to this hydrophobic portion tends to increase the water solubility of the molecule as a whole, and the liquid character of the product is retained up to the point where the polyoxyethylene content is about 50% of the total weight of the condensation product, which corresponds to condensation with up to about 40 moles of ethylene oxide. Examples of compounds of this type include certain of the commercially-available Pluronic® surfactants, marketed by BASF, and Antarox, marketed by Rhodia.

The condensation products of ethylene oxide with the product resulting from the reaction of propylene oxide and ethylenediamine. The hydrophobic moiety of these products consists of the reaction product of ethylenediamine and excess propylene oxide, and generally has a molecular weight of from about 2500 to about 3000. This hydrophobic moiety is condensed with ethylene oxide to the extent that the condensation product contains from about 40% to about 80% by weight of polyoxyethylene and has a molecular weight of from about 5,000 to about 11,000. Examples of this type of nonionic surfactant include certain of the commercially available TetronicB compounds, marketed by BASF.

Semi-polar nonionic surfactants are a special category of nonionic surfactants which include water-soluble amine oxides containing one alkyl moiety of from about 10 to about 18 carbon atoms and 2 moieties selected from the group consisting of alkyl groups and hydroxyalkyl groups containing from about 1 to about 3 carbon atoms; water-soluble phosphine oxides containing one alkyl moiety of from about 10 to about 18 carbon atoms and 2 moieties selected from the group consisting of alkyl groups and hydroxyalkyl groups containing from about 1 to about 3 carbon atoms; and water-soluble sulfoxides containing one alkyl moiety of from about 10 to about 18 carbon atoms and a moiety selected from the group consisting of alkyl and hydroxyalkyl moieties of from about 1 to about 3 carbon atoms.

Semi-polar nonionic detergent surfactants include the amine oxide surfactants. These amine oxide surfactants in particular include C₁₀-C₁₈ alkyl dimethyl amine oxides and C₈-C₁₂ alkoxy ethyl dihydroxy ethyl amine oxides.

Alkylpolysaccharides disclosed in U.S. Pat. No. 4,565,647, Llenado, issued Jan. 21, 1986, having a hydrophobic group containing from about 6 to about 30 carbon atoms, preferably from about 10 to about 16 carbon atoms and a polysaccharide, e.g., a polyglycoside, hydrophilic group containing from about 1.3 to about 10, preferably from about 1.3 to about 3, most preferably from about 1.3 to about 2.7 saccharide units. Any reducing saccharide containing 5 or 6 carbon atoms can be used, e.g., glucose, galactose and galactosyl moieties can be substituted for the glucosyl moieties. (Optionally the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units.

Optionally, and less desirably, there can be a polyalkylene-oxide chain joining the hydrophobic moiety and the polysaccharide moiety. The preferred alkyleneoxide is ethylene oxide. Typical hydrophobic groups include alkyl groups, either saturated or unsaturated, branched or unbranched containing from about 8 to about 18, preferably from about 10 to about 16, carbon atoms. Preferably, the alkyl group is a straight chain saturated alkyl group. The alkyl group can contain up to about 3 hydroxy groups and/or the polyalkyleneoxide chain can contain up to about 10, preferably less than 5, alkyleneoxide moieties. Suitable alkyl polysaccharides are octyl, nonyl, decyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses and/or galactoses. Suitable mixtures include coconut alkyl, di-, tri-, tetra-, and pentaglucosides and tallow alkyl tetra-, penta-, and hexa-glucosides.

The preferred alkylpolyglycosides have the formula:
R²O(CₙH₂ₙO)ₜ(glycosyl)ₓ
wherein R² is selected from the group consisting of alkyl, alkyl-phenyl, hydroxyalkyl, hydroxyalkylphenyl, and mixtures thereof in which the alkyl groups contain from about 10 to about 18, preferably from about 12 to about 14, carbon atoms; n is 2 or 3, preferably 2; t is from 0 to about 10, preferably 0; and x is from about 1.3 to about 10, preferably from about 1.3 to about 3, most preferably from about 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1position and the preceding glycosyl units 2-, 3-, 4- and/or 6-position, preferably predominantly the 2-position.

Non ionic surfactant include fatty acid amide surfactants having the formula: 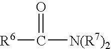
wherein R⁶ is an alkyl group containing from about 7 to about 21 (preferably from about 9 to about 17) carbon atoms and each R⁷ is selected from the group consisting of hydrogen, C₁-C₄ alkyl, C₁-C₄ hydroxyalkyl, and —(C²H₄O)ₓH where x varies from about 1 to about 3. Preferred amides are C₈-C₂₀ ammonia amides, monoethanolamides, diethanolamides, and isopropanolamides.
Cationic Surfactants

Cationic detersive surfactants can also be included in detergent compositions of the present invention. Cationic surfactants include the ammonium surfactants such as alkyldimethylammonium halogenides, and those surfactants having the formula: [R²(OR³)_{y}]R⁴(OR³)_{y}]₂R⁵N⁺X⁻ wherein R² is an alkyl or alkyl benzyl group having from about 8 to about 18 carbon atoms in the alkyl chain, each R³ is selected from the group consisting of —CH₂CH₂—, —CH₂CH(CH₃)—, —CH₂CH(CH₂OH)—, —CH₂CH₂CH₂—, and mixtures thereof; each R⁴ is selected from the group consisting of C₁-C₄ alkyl, C₁-C₄ hydroxyalkyl, benzyl, ring structures formed by joining the two R⁴ groups, —CH₂CHOHCHOHCOR⁶CHOH—CH₂OH wherein R⁶ is any hexose or hexose polymer having a molecular weight less than about 1000, and hydrogen when y is not 0; R⁵ is the same as R⁴ or is an alkyl chain wherein the total number of carbon atoms of R² plus R⁵ is not more than about 18; each y is from 0 to about 10 and the sum of the y values is from 0 to about 15; and X is any compatible anion.

Other cationic surfactants useful herein are also described in U.S. Pat. No. 4,228,044, Cambre, issued Oct. 14, 1980, incorporated herein by reference.

Other Surfactants

Ampholytic surfactants can be incorporated into the detergent compositions hereof. These surfactants can be broadly described as aliphatic derivatives of secondary or tertiary amines, or aliphatic derivatives of heterocyclic secondary and tertiary amines in which the aliphatic radical can be straight chain or branched. One of the aliphatic substituents contains at least about 8 carbon atoms, typically from about 8 to about 18 carbon atoms, and at least one contains an anionic water-solubilizing group, e.g., carboxy, sulfonate, sulfate. See U.S. Pat. No. 3,929,678 to Laughlin et al., issued Dec. 30, 1975 at column 19, lines 18-35 for examples of ampholytic surfactants. Preferred amphoteric include C₁₂-C₁₈ alkyl ethoxylates (“AE”) including the so-called narrow peaked alkyl ethoxylates and C₆-C₁₂ alkyl phenol alkoxylates (especially ethoxylates and mixed ethoxy/propoxy), C₁₂-C₁₈ betaines and sulfobetaines (“sultaines”), C₁₀-C₁₈ amine oxides, and mixtures thereof

Zwitterionic surfactants can also be incorporated into the detergent compositions hereof These surfactants can be broadly described as derivatives of secondary and tertiary amines, derivatives of heterocyclic secondary and tertiary amines, or derivatives of quaternary ammonium, quaternary phosphonium or tertiary sulfonium compounds. See U.S. Pat. No. 3,929,678 to Laughlin et al., issued Dec. 30, 1975 at column 19, line 38 through column 22, line 48 for examples of zwitterionic surfactants. Ampholytic and zwitterionic surfactants are generally used in combination with one or more anionic and/or nonionic surfactants.

Other optional ingredients of the composition include:

thickening polymers,

hydrophilizing polymers,

soil-release polymers,

deposition agents or deposition aid agents, for example deposition polymers, such as silicones,

anti-foaming agents,

foaming agents,

foam stabilizing and/or enhancing agents,

perfumes or fragrances,

builders (detergency adjuvants), including organic builders and inorganic builders

buffers,

salts or fillers,

chelatants,

colorants,

preservatives,

enzymes,

corrosion inhibitors,

scale inhibitors,

dyes,

optical bighteners,

soiling suspending agents,

freezing-thawing stabilizers,

solvents,

opacifiers,

pearlescence agents.

According to some advantageous embodiments, the composition is:

a fabric care composition

a carpet cleaning composition, for example a carpet cleaning spray, foam, or foaming liquid,

a softening and/or antiwrinkling and/or easy-ironing composition,

a laundry product in a liquid form, powder form or tablet form, that can be use in hand washing or in automatic washing,

a hand dishwashing composition,

an automatic dishwashing composition, in a liquid form, in a powder form, or in a tablet form,

a glass cleaning composition, including a window cleaning composition or a car windshield composition,

a floor cleaning composition,

a versatile cleaning hard surface cleaning compostion,

a kitchen cleaning composition,

a bathroom cleaning composition,

a toilet bowl cleaning composition, a rim block, a rim liquid

a wood treatment composition,

a shower rinse (daily shower),

a spray for providing disinfection and/or suppression of odors and/or prevention of malodor for clothes, curtains, sheets, furniture fabrics or car fabrics,

a car cleaning composition,

an air freshener,

a tile or plastic floor cleaning composition.

According to one embodiment:

the surface is a fabric surface,

the composition is fabric-care composition,

the composition is applied onto the surface during a washing process, preferably automatic process:
optionally applying a pre-spotting composition onto the fabric surface,
washing with a detersive composition,
rinsing with water optionally comprising a rinsing composition,
optionally further rinsing,

the composition being the pre-spotting composition, the detersive composition, or the rinsing composition.

In this embodiment, the composition can be a rinsing composition such as a softening and/or antiwrinkiing and/or easy-ironing composition.

In this embodiment, the composition can also be a detersive laundry-product composition in a liquid form, in a powder form, or in a tablet form, optionally being a multiple benefit composition providing cleaning and softening and/or antiwrinkiing and/or easy-ironing.

In another embodiment:

the surface is a fabric surface, for example clothes, curtains, sheets, furniture fabrics or car fabrics, and

the composition is applied onto the surface by spraying.

In this embodiment, the composition can be a spray used on a regularly basis or to prevent malodors and/or to suppress them, such as for example some spray compositions marketed as “Frebreze” by the Procter and Gamble company. The composition can also be used in ironing (“starching” compositions or “ironining moisturizing” compositions).

In another embodiment:

the surface is a carpet, and

the composition is a carpet cleaning composition, in the form of a liquid, a foaming liquid, a spray, or a spraying composition,

the composition is applied onto the carpet, the carpet is clean, and then optionally rinsed.

Cleaning the carpet, can be performed by any mean, for example by washing and/or shampooing.

According to another embodiment:

the surface is a hard surface,

the composition is applied onto the surface by a process comprising the following step:
optionally diluting the composition,
applying the composition optionally diluted
optionally rinsing.

The composition can by applied onto the surface by any appropriate mean. The applying processes and means can depend upon the surface the be treated, and upon the user.

The surface in this embodiment can be:

glass, for example comprised in windows, or cars windshields,

tile or ceramic, for example comprised in kitchen, bathroom, toilet bowls, showers, china, dishware, floors,

metal, for example comprised in silverware, car body parts, window frames, or floors

plastic, for example comprised in dishware, silverware, cars, windows, furniture, or floors

wood or leather, optionally waxed, for example comprised in furniture or floor,

concrete, optionally waxed, for example comprised in floors.

In hand dishwashing, the composition can be applied by immersing the dishware into a diluted composition, by applying a concentrate composition directly onto the dishware, or by applying the composition onto a cleaning device such as a sponge, cloth, pad, scrub, brush or any other mean.

In automatic dishwashing, the compositions can be applied in diluted form through conventional automatic dishwashing schemes.

In windows, floors, furniture or any other hard surface different from dishware or silverware, the composition can be applied by any conventional mean, including direct spraying, spraying after dilution, application with using a vehicle such as mops, pads, wipes, cloths, sponges, papers

In a more general view, the invention can find applications and/or to be used in the following:

Home-Care, Industrial and Institutional Cleaning, including (but not limited to) the following:
Laundry and textile care, including detergent compositions (automatic or hand wash), softeners formulations or products, anti-wrinkling formulations or products,
Dish-washing (automatic or hand),
Hard Surface Cleaning: including car wash, windows cleaning (buildings or cars), toilet bowls cleaning, shower rinse, domestic and industrial surfaces wash or cleaning,

Personal-Care, including (but not limited to) the following:
rinse-off products, including shampoos, shower gels, conditioning formulations, after shampoos, personal cleansing, hair-coloration products,
leave-on products, including make-up, after-shampoos, skin-care products such as creams, milks, hair styling products (gels, mousses, sprays), color cosmetics,
sun-protection products,

Baby-Care, Wipes, Female Care, including (but not limited to) the following:
diapers surface treatments,
dry wipes, wet wipes,

Oral Care formulations, including (but not limited to) the following:
Toothpaste,
Whitening,
Oral rising,

Industrial Markets, including (but not limited to) the following:
food or non-food industrial workshop cleaning
industrial surfaces cleaning, including industrial metal cleaning (panels, plates, sheets)
lubricants

Oilfield formulations

Paints and coatings, including (but not limited to) the following:
paints, boat paints (anti-fouling),
Anti-dirt and anti-soil paints (decorative and exterior),
Plastic adhesion promoters (industrial, decorative and exterior),
Metal adhesion promoters (decorative and industrial paints),
Mortars and Adhesives: Plastic adhesion promoters. Glass and ceramic adhesion promoters. PSA.
Sealants: Plastic adhesion promoters. Glass and ceramic adhesion promoter,
Textile coating, fabrics treatments or fibers treatments, hydrophilisation, hydrophobisation, breathability, anti-soil, anti dirt treatments
Technical plastics: surfaces modifiers (hydrophilisation and functionnalisation),
Industrial and Water treatment: protective coatings against corrosion and bacteria
Adhesion promoters,
Rubber reinforcement: Metal adhesion promoters, Plastic adhesion promoters.

Food Technology including (but not limited to) the following:
process equipment treatments (foods&drinks, incl. potable water),
filtration,
heat exchange,
emulsification,
membranes (for preparing potable water),
food preparation industrial workshops,
food handling equipment,
food packaging,

Biomedical materials & devices, health care including (but not limited to) the following:
contact lenses,
blood dialysis,
materials to be used in contact with tissue & biofluids,
catheters,
blood bags,
prostheses,
implants for cardiovascular surgery,
voice prostheses,
stents,
dental surgery,
medical utensils,

Crop protection including (but not limited to) the following:
Anti-injection agents,
Seeds coating.

The following examples further illustrate the present invention without limiting the scope of the appended claims.

### EXAMPLE 1

Poly(N-methyl-4-vinylpyridiniumjodide (PVP) was used as obtained from Polymer Source Inc. (Montreal, Canada). 0.5 g PVP was dissolved in 10 mM aqueous solution of NaNO₃ to a concentration of approximately 0.5 g/l. Separately, two batches with poly(acrylic acid)-co-poly(acryl amide) under the name of PAA42-PAM97 and PAA42-PAM417 were prepared by dissolution of 0.5 g PAA42-PAM97 and 0.5 g PAA42-PAM417 in 10 mM NaNO₃-solutions to concentrations of about 0.5 g/l. and 1.5 g/l. respectively.

PAA42-PAM97 is a diblock copolymer comprising a block having about 42 units deriving from acrylic acid, and a block having about 97 units deriving from acrylamide. PAA42-PAM417 is a diblock copolymer comprising a block having about 42 units deriving from acrylic acid, and a block having about 97 units deriving from acrylamide. The diblock copolymers can be obtained for example by process substantially described in document ???? with adjusting the amounts of monomers used.

The fraction of cationic groups in PVP and the fraction of anionic groups in the block polymers PAA42-PAM97 and PAA42-PAM417 were calculated from the numbers as provided by the manufacturers, see table 1. The polydispersities of all polymers were low, typically around 1.05-1.1. All other chemicals (salts, basic and acidic solutions) were of analytical grade.

TABLE 1
Block lengths and molecular weight of PAA-PAM and PVP

| | no. of ionic | no. of neutral | |
|---|---|---|---|
| | monomers | monomers | MW (g/mol) |
| PAA42-PAM97 | 42 | 97 | 10000 |
| PAA42-PAM417 | 42 | 417 | 33000 |
| PVP | 209 | | 56000 |

The formation of complex coacervate core micelles was now studied by dynamic light scattering upon mixing the PAA42-PAM97- and PAA42-PAM417-batches of various compositions with the PVP-solution in a light scattering cell. Dynamic light scattering was performed with an ALV light scattering instrument equipped with a 400 mW argon ion laser tuned at a wavelength of 514 nm. For each mixture the autocorrelation function was recorded, which arises from the Brownian motion of particles in a liquid, which in case of the detection of particles yielded a diffusion coefficient, that was converted by the Stokes-Einstein equation into a hydrodynamic particle radius.

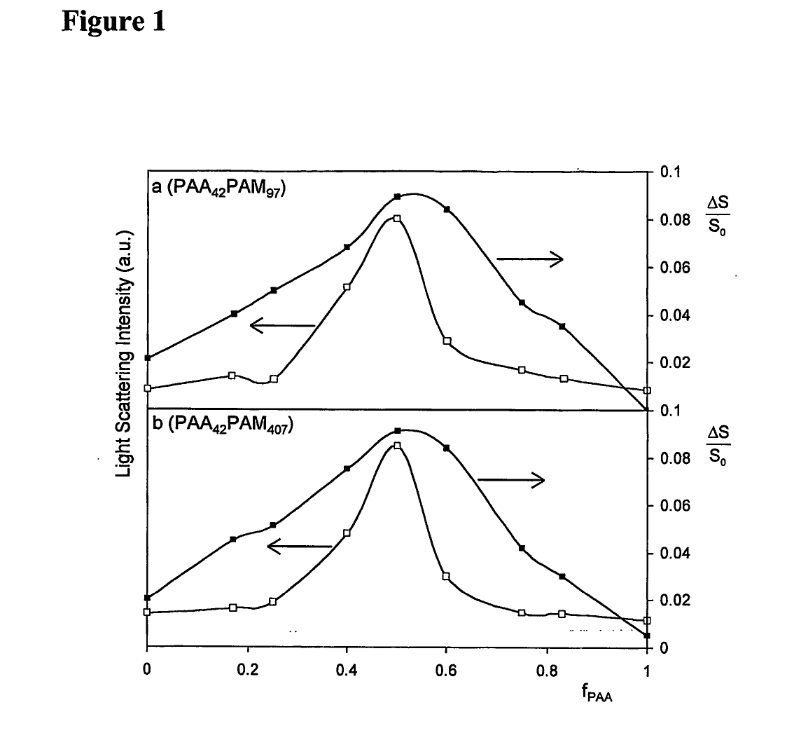

The light scattering results were plotted in FIG. 1 (open squares) for mixtures of PAA42-PAM97 with PVP (top) and PAA42-PAM417 with PVP (bottom) as a function of the fraction f⁻ of anionic PAA-groups over the total number of charged groups (from both PAA and PVP). Along the y-axis the light scattering data were plotted as measured intensities. Only at approximately f=0.5 particles were detected.

For both batches of block polymer the maximum size was reached when f⁻ is about half, meaning that the amount of anionic and cationic groups were roughly equal. The hydrodynamic radii at these concentrations were approximately 20 and 25 nm with PAA42-PAM97 and PAA42-PAM417, respectively.

### EXAMPLE 2

The titration experiment of example 1 was repeated with the batches of 10 mM NaNO₃ solutions of PAA42-PAM97, PAA42-PAM417 and PVP as prepared therein. Now the adsorption from these solutions onto a hydrophilic surface was monitored by reflectometry as a function of the fraction of anionic groups. As the surface a silicon wafer (Aurel GmbH, Germany) was applied, carrying an oxide-layer of about 73 nm (as determined by ellipsometry). The pH in these solutions was adjusted to pH 7 using NaOH and HNO₃ solutions. At this pH the silica surface is negatively charged.

The adsorption conditions and the conversion of raw reflectometry data in to the adsorbed amount are described in “Reflectometry as a tool for adsorption studies”, Adv. Colloid Interface Sci. 1994, vol. 50, p. 79-101. The base reflectivity signal S₀ was first determined flushing 10 mM NaNO₃ through the cell in which the monitored surface was exposed to the solution that was pumped through. The change in reflectivity upon switching to a solution containing a particular mixture of PAA-PAM and PVP was followed until a stable signal S was reached again, corresponding to maximal adsorption. The reflectivity over the reflectivity before addition of the adsorbate S/S₀, which is linearly proportional with the adsorbed amount, was determined for different amounts of anionic groups in the solution and plotted together with the light scattering data in FIG. 1 (filled squares).

The results were in agreement with the previous example, showing that maximal adsorption was reached at roughly equal amounts of PAA- and PVP-groups in the composition. The maximum values corresponded to 3 mg/m² for both batches. The pure components PAA42-PAM97 and PAA42-PAM417 barely adsorbed on the silica surface (f=1), and the adsorption of pure PVP (f=0) was also far behind the levels reached when the fractions of anionic and cationic groups in the solution were comparable.

When exposing the layers to background solvent again, i.e. 10 mM NaNO₃ solution, only a few percent of the adsorbed mass was rinsed from the surface. After this quick desorption step, a stable plateau value was obtained again. When the layers were again exposed to the micellar solutions, the adsorbed mass returned to its original plateau value. Upon exposure to 1 M NaNO₃ solution, the adsorbed mass decreased to approximately 80% of the original plateau value, corresponding to a maximal adsorbed amount of 3 mg/m² for PAA42-PAM97 and PVP, with f=0.5.

### EXAMPLE 3

0.5 g PAA42-PAM97 and 0.5 g PVP were dissolved in 2 1 of a 0.10 mM NaNO₃-solution, yielding f=0.5. The pH was adjusted to 7 using NaOH and HNO₃ solutions. The polydispersities of the polymers were low, typically around 1.05-1.1. The chemicals (salts, basic and acidic solutions) were of analytical grade.

Adsorption from these solutions was according to example 2, but now applying a polystyrene coated silica surface layer thickness 66 nm. The molecular weight of the polystyrene, available by the name of P630-St from Polymer Source Inc. (Montreal, Canada) was 850 K. The robustness of the adsorbed layer is tested by exposure to solvent and 1 M NaNO₃-solution.

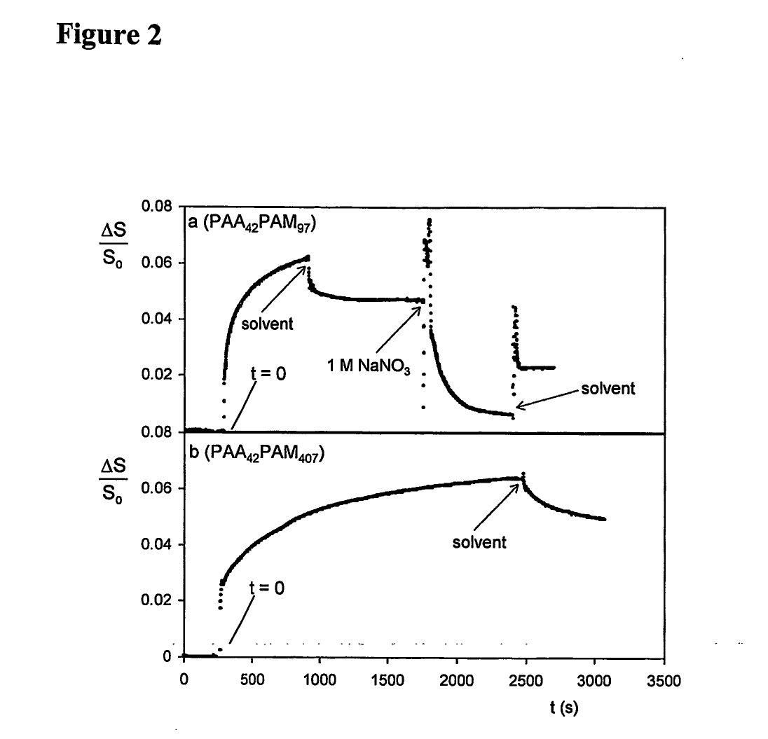

The evolution of the relative adsorption signals S/S₀ is shown in FIG. 2, 0.1 S/S₀-unit corresponding with 4 mg/m². FIG. 2 shows that the micelles also adsorbed onto this hydrophobic surface at 0.01 M NaNO₃. The exposure to solvent eroded the layers to about two-third of the original signal.

With the addition of 1 M NaNO₃-solution the layer eroded even further. The large fluctuations in the signal resulted from large differences in refractive index between 10 mM and 1 M NaNO₃, that created a lot of scattering when the liquids were mixed in the reflectometry cell. After a short time, however, a steady signal was obtained again, yielding a plateau value at roughly one-third of the primary adsorption plateau value. Therefore both solvent and 1 M NaNO₃ were not able to completely erode the layer.

### EXAMPLE 4

The adsorption of lysozyme on a silica surface, either uncoated or coated with solutions of PAA42-PAM97/PVP and PAA42-PAM417/PVP with f=0.5, was studied using reflectometry according to example 2. Thereto lysozyme from chicken egg white [12650-88-3], obtained from Sigma (L6876), lot. 51K7028, was dissolved in 10 mM NaNO₃-solution to a concentration of 100 ppm. The pH was set at about 7 with NaOH solution.

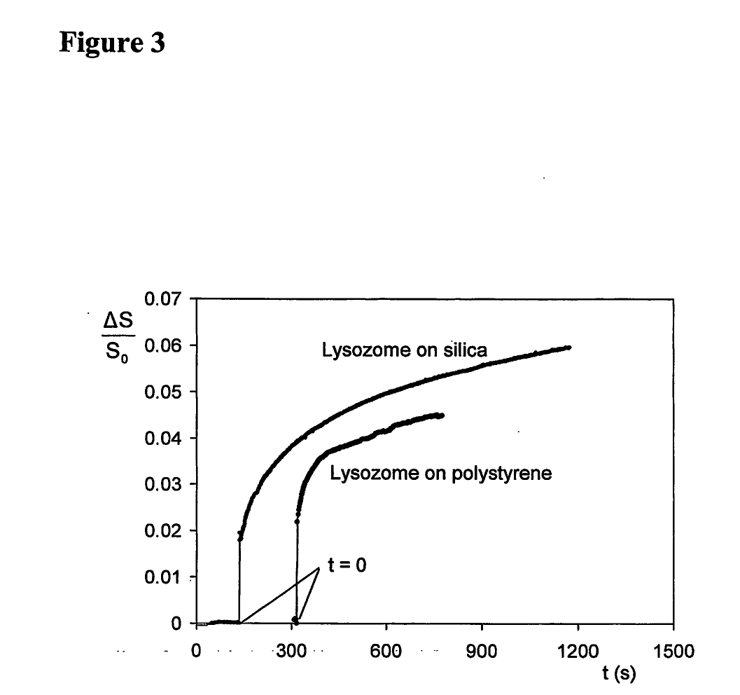

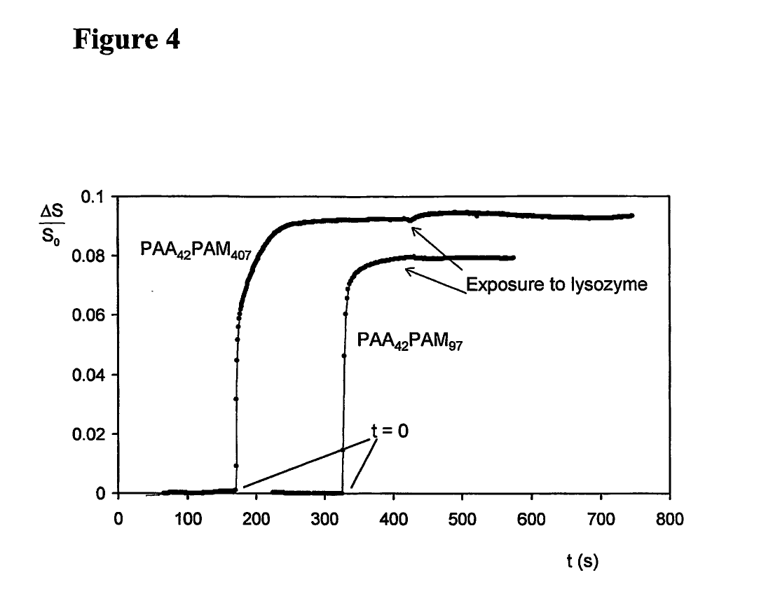

FIG. 3 shows the adsorption of lysozyme directly onto a bare silica surface in terms of relative reflectivity S/S₀, and FIG. 4 presents the evolution of the adsorption from the same solution, but now only after coating of the silica surface with either a f=0.5 solution of PAA42-PAM97/PVP or PAA42-PAM417/PVP, as described in example 2. After reaching steady adsorption and before lysozyme addition the reflectometry cell was first flushed with background solvent (10 mM NaNO₃) until a stable background signal was obtained.

From the FIGS. 3 and 4 it can be concluded that the adsorption of lysozyme on this hydrophilic surface was prohibited by the layers of polymeric micelles of PAA-PAM and PVP.

### EXAMPLE 5

The variation of the corona block length was investigated by mixing diblock copolymers of polyacrylic acid (PAA) and polyacrylamide (PAM) with a poly-(dimethylamino ethylmethacrylate) (PAMA) homopolymer, that was synthesized according to the method described in Hoogeveen et al. “Novel water-soluble block copolymers of dimethylaminoethyl methacrylate and dihydroxypropyl methacrylate”, Macromolecular Chemical Physics, 1996, vol. 197, pages 2553-2564. The length of the neutral polyacrylamide-block in the diblock copolymer was an adjustable parameter.

The different batches of PAA-PAM block copolymers were used in a concentration of 2.7 mM of PAA-monomers in 30 mM NaNO₃. These concentrations were determined on the basis of the specifications given by the manufacturer (see table 2). The polydispersities of all polymers were low, typically around 1.05-1.1, the chemicals are of analytical grade.

These diblock copolymers were titrated with PAMA according to example 1. The obtained aqueous mixtures, characterised by the fraction of PAMA-monomers f_{PAMA}, were studied with dynamic light scattering.

When f_{PAMA} was between 0.3 to 0.6, particles with hydrodynamic radii of 20 to 40 nm were detected, indicating the formation of complex coacervate core micelles. The measured radii as a function of the monomeric PAM-units showed a linear dependence, yielding the largest particle size for the block of 417 PAM-units.

TABLE 2
Variation in block length of neutral block in
PAA-PAM diblock copolymer

| | no. of ionic | no. of neutral | |
|---|---|---|---|
| | monomers | monomers | MW (g/mol) |
| PAA42-PAM42 | 42 (acylic acid) | 42 (acrylamide) | 6,000 |
| PAA42-PAM97 | 42 (acylic acid) | 97 (acrylamide) | 10,000 |
| PAA42-PAM208 | 42 (acylic acid) | 208 (acrylamide) | 18,000 |
| PAA42-PAM417 | 42 (acylic acid) | 417 (acrylamide) | 33,000 |
| PAMA | 126 | | |

### EXAMPLE 6

In a DLS-titration study according to example 5 the effect of the molecular weight of the second polymer on the polymeric micelles according the invention was investigated. Thereto, 1 g/l aqueous PAMA35-PGMA105-solution was mixed with aqueous solutions of PMA with a chain length of 1300 and 6900 monomers (respective molecular weight of 113 and 600 K). PAMA35-PGMA105 was synthesized according to the method described in Hoogeveen et al. “Novel water-soluble block copolymers of dimethylaminoethyl methacrylate and dihydroxypropyl methacrylate”, Macromolecular Chemical Physics, 1996, vol. 197, pages 2553-2564. The concentration of PMA-monomeric units the diblock copolymer was titrated with was 17 mM. The ionic strength was 100 mM NaCl throughout the experiment.

When the fraction of PMA monomers was between approximately 0.4 and 0.6, micelles with hydrodynamic radii of about 15 and 25 nm were detected for PMA1300 and PMA6900, respectively.

### EXAMPLE 7

According to example 6, only using PAMA12-PGMA118 instead of PAMA35-PGMA105 and PAA with 2100 monomeric units (molecular weight 154 K) instead of PMA. The diblock copolymer was dissolved to a concentration of 5 g/l in 100 mM NaNO₃, and then titrated with 33 mM PAMA-monomeric units (in 100 mM NaNO₃).

Again, when the fraction of PAA monomers was between approximately 0.4 and 0.6, micelles with a radius of about 30 nm size were observed.

1-34. (canceled)

35. A process for the modification or the treatment of a surface, comprising the step of coating said surface with a composition comprising at least one polymeric micelle, the polymeric micelle having a hydrophilic, neutral corona and a complex coacervate core, said complex coacervate core being formed by charge complexation.

36. The process according to claim, wherein the surface modification or surface treatment is for rendering at least one surface of a device protein-resistant.

37. The process according to claim 36, wherein the surface modification or surface treatment is for preventing bacteria proliferation, disinfecting, suppressing odours, preventing malodour, or for providing easy-cleaning or soil-release properties.

38. The process according to claim 35, wherein the polymeric micelle comprises at least a first and a second polymer.

39. The process according to claim 38, wherein the first polymer and the second polymer are oppositely charged.

40. The process according to claim 39, wherein the first polymer is a block polymer with an ionic block comprising at least 6 chargeable groups.

41. The process according to claim 40, wherein the ionic block is selected from the group consisting of polyacrylic acid, polymethacrylic acid, poly-(dimethylamino ethylmethacrylate) and poly(N-alkyl-4-vinylpyridinium).

42. The process according to claim 38, wherein the first polymer comprises at least a hydrophilic and neutral block.

43. The process according to claim 42, wherein the hydrophilic and neutral block is a polyethylene glycol or a polyacrylamide, or a combination thereof.

44. The process according to claim 38, wherein the second polymer is a homopolymer, a random copolymer, a block polymer, a natural polymer, or a derivative thereof.

45. The process according to claim 44, wherein the homopolymer is selected from the group of polyacrylic acid, polymethacrylic acid, poly-(dimethylamino ethylmethacrylate) and poly(N-alkyl-4-vinylpyridinium).

46. The process according to claim 35, for rendering at least one surface of a device protein-resistant, for the reduction or prevention of protein adsorption and/or anti-fouling.

47. The process according to claim 35, for preventing bacteria proliferation, disinfecting, suppressing odours, preventing malodour, for providing easy-cleaning or soil-release properties, wherein the coating composition is a home-care or fabric-care or institutional-cleaning or industrial-cleaning composition.

48. A process for modifying a surface or treating a surface, said process comprising the steps of:
(i) mixing at least a first and a second polymer in such amounts that the resulting mixture has a fraction of the total number of cationic polymeric groups over the total number of charged groups in the range of 0.2 to 0.8, wherein the first and the second polymer are oppositely charged and wherein the first polymer is a block polymer comprising at least a hydrophilic and neutral block; and
(ii) bringing the resulting mixture under aqueous conditions in contact with the surface,
wherein the salt concentration in both steps is less than 1 M.

49. The process according to claim 48, wherein the first polymer is a block polymer with an ionic block comprising at least 6 chargeable groups.

50. The process according to claim 49, wherein the ionic block is selected from the group consisting of polyacrylic acid, polymethacrylic acid, poly-(dimethylamino ethylmethacrylate) and poly(N-alkyl-4-vinylpyridinium).

51. The process according to claim 50, wherein the hydrophilic and neutral block is a polyethylene glycol, a polyglycerylmethacrylate or a polyacrylamide, or a combination thereof.

52. The process according to claim 48, wherein the second polymer is a homopolymer a random copolymer, a block polymer, a natural polymer, or a derivative thereof.

53. The process according to claim 52, wherein the homopolymer is selected from the group consisting of polyacrylic acid, polymethacrylic acid, poly-(dimethylamino ethylmethacrylate) and poly(N-alkyl-4-vinylpyridinium).

54. A modified surface or treated surface comprising a coated surface, wherein the coated surface comprises at least one polymeric micelle immobilized to the surface, wherein the polymeric micelle has a charged core and a hydrophilic, neutral corona.

55. The modified surface or treated surface according to claim 54, wherein the polymeric micelle comprises a first and a second polymer and, wherein the first polymer and the second polymer are oppositely charged.

56. The modified surface or treated surface according to claim 55, wherein the ionic block is selected from the group consisting of polyacrylic acid, polymethacrylic acid, poly-(dimethylamino ethylmethacrylate) and poly(N-alkyl-4-vinylpyridinium).

57. The modified surface or treated surface according to claim 56, wherein the first polymer comprises at least a hydrophilic and neutral block.

58. The modified surface or treated surface according to claim 57, wherein the hydrophilic and neutral block is a polyethylene glycol, a polyglycerylmethacrylate or a polyacrylamide, or a combination thereof.

59. The modified surface or treated surface according to claim 55, wherein the second polymer is a homopolymer selected from the group consisting of polyacrylic acid, polymethacrylic acid, poly-(dimethylamino ethylmethacrylate) and poly(N-alkyl-4-vinylpyridinium).
